(19) United States
(12) Patent Application Publication
(10) Pub. No.: US 2024/0132931 A1
(43) Pub. Date: Apr. 25, 2024

(54) Biological Indicator with Enhanced Volatile Organic Compound Detection

(71) Applicant: O&M Halyard, Inc., Mechanicsville, VA (US)

(72) Inventors: Anthony S. Spencer, Woodstock, GA (US); Bharadwaja Srimat Tirumala Peddinti, Houston, TX (US); Sylvie Dufresne, Quebec (CA)

(21) Appl. No.: 18/478,096

(22) Filed: Sep. 29, 2023

Related U.S. Application Data
(60) Provisional application No. 63/411,976, filed on Sep. 30, 2022.

(51) Int. Cl. C12Q 1/04

(52) U.S. Cl. CPC C12Q 1/045

(57) ABSTRACT

A growth medium for a biological indicator is provided. The growth medium can include a base growth medium as well as an additive. The additive can include a carbon source unit, such as, but not limited to, glucose, and a volatile organic compound unit. Alternatively, the additive can include a carbohydrate source. The present invention is also directed to a self-contained biological indicator (SCBI) that includes a container, spores disposed on a carrier, a growth medium, and an additive. The additive can include a carbon source unit or a molecule containing a part that can form a VOC when reduced or oxidized, such as, but not limited to glucose, and a volatile organic compound unit. Alternatively, the additive can include a carbohydrate source. It has been found that the addition of such additives to the growth medium facilitates the efficient and accurate detection of a failed sterilization process.

### RELATED APPLICATIONS

The present application claims priority to U.S. Provisional Patent Application No. 63/411,976, filed Sep. 30, 2022, the entire contents of which are incorporated herein by reference.

### FIELD OF THE INVENTION

The present invention relates generally to a growth medium for a biological indicator used to verify the effectiveness of a sterilization process.

### BACKGROUND

In medical or pharmaceutical sterilization processing, achieving, and maintaining sterility is of utmost importance. A biological indicator (BI) is often used to assess the lethality of sterilization process in such cases. A biological indicator typically includes the most resistant endospores, or bacterial spores, of a defined strain for the specific sterilization process. For example, Geobacillus stearothermophilus spores are typically used in biological indicators to assess the effectiveness of the process during a steam (gravity or dynamic air-removal) or oxidative sterilization process such as hydrogen peroxide, while Bacillus atrophaeus spores are typically used in biological indicators to assess the effectiveness of the processing during an ethylene oxide sterilization cycle. However, it is important that the detection sensitivity of the measurement be accurate enough to identify even if just one or two spores are not killed after the sterilization cycle to avoid a false negative result. Currently available biological indicators rely on various techniques to identify if spore biological activity is still present post-sterilization. In the first generation of biological indicators, failure was measured by turbidity of the growth medium. Meanwhile, second generation biological indicators are self-contained systems which include the spore strip and growth medium required for recovery in a primary pack that is ready for use. These biological indicators rely on a pH indicator to measure the production of acid metabolites in the growth medium by outgrowing spores and replicating cells. Next, third-generation biological indicators have a dual readout system. The rapid portion detects active spore-associated α-glucosidase enzyme surviving the sterilization process within a particular time frame, as specified by the manufacturer of the biological indicator and reader. The enzyme is a normal constituent of vegetative cells and spores of Geobacillus stearothermophilus and Bacillus atrophaeus. G. stearothermophilus spores have significant amounts of associated α-glucosidase activity due to at least two enzymes. Some of this activity is due to an enzyme on the spore's outer surface and in the initial dormant spore core, and much is also due to the enzyme synthesized during spore germination and outgrowth. B. atrophaeus does not have such enzymatic activity in the spore, and the activity is induced during germination. Activity of the enzyme present in the outer membrane and cortex stop after 20 minutes of the start of the germination. Thereafter, it is the inner membrane enzymes that are active.

The survival of the spore associated α-glucosidase enzyme following exposure to steam sterilization does not correlate well with spore survival. This is because regular spores (not genetically modified) do not contain enough of the enzyme for a low number of active spores to be detected via fluorescence. Where the microorganism is Geobacillus stearothermophilus or Bacillus atrophaeus, the number of microorganisms necessary to produce a sufficient level of the enzyme to detect the fluorescence is about 1×10³ to 1×10⁸ microorganisms. To increase sensitivity, spore genetic material can be modified, or additional enzyme extract can be added to the biological indicator. As part of the third generation, in some cases, there is a second readout system that can include a pH indicator, which detects acid metabolites produced by outgrowing spores and replicating cells and gives confirmation of the rapid result within a particular time frame, as specified by the manufacturer of the biological indicator and reader. With each generation of biological indicators, the detection time decreases, but there continues to be a need to drive the response time to as close to zero as possible.

Volatile organic compounds (VOCs) have been used as markers for endospores or bacterial spore germination and growth. VOC measurement involves the use of various detection methods found in the published literature and art. The concentration of the VOCs captured directly corresponds to the germination activity of spores. However, it is critical that the detection sensitivity of the VOCs be capable of identifying the minimum threshold level of endospores or bacterial spore VOCs that indicate failure of sterilization kill. Therefore, there is a need to have an endospore or bacterial spore and growth medium combination that produces detectable level of VOCs to avoid a biological indicator that outputs false negative readings post sterilization.

### SUMMARY OF THE INVENTION

Objects and advantages of the invention will be set forth in part in the following description, or may be obvious from the description, or may be learned through practice of the invention.

In one embodiment, a growth medium for a biological indicator is provided. The growth medium includes a base growth medium as well as an additive comprising a carbon source unit, such as, but not limited to, glucose, and a volatile organic compound unit. In another embodiment, a self-contained biological indicator (SCBI) can include a container, spores disposed on a carrier, a growth medium, and an additive. The additive can be contained within the growth medium, can be disposed on the carrier, or can be disposed on the spores themselves. The additive includes a carbon source unit such as, but not limited to a carbohydrate or amino acid molecules; or a carbon source molecule containing a part that can form a VOC when reduced or oxidized, such as, but not limited to glucose, and a volatile organic compound unit.

In still another embodiment, a growth medium for a biological indicator is provided. The growth medium includes a base growth medium; and an additive that includes a carbohydrate source that leads to formation of a volatile organic compound by an action of an enzyme or a coenzyme that is active in a germination phase of spores that are introduced to the growth medium. For example, that additive can include an amino acid. For instance, the additive can include an aliphatic amine. The release of cytochrome c reductase as a result of spores that have been introduced to the growth medium being in the germination phase can result in an oxidation reaction to yield the volatile organic compound. Further, the volatile organic compound can include an aldehyde.

In yet another embodiment, a self-contained biological indicator is provided. The SCBI includes a container; spores disposed on a carrier; and a growth medium, wherein the self-contained biological indicator includes a carbohydrate source that leads to formation of a volatile organic compound by an action of an enzyme or a coenzyme that is active in a germination phase of spores that are introduced to the growth medium. The carbohydrate source can be contained within the growth medium, can be disposed on the spore carrier, or can be disposed on the spores themselves. The release of cytochrome c reductase as a result of the spores that have been introduced to the growth medium being in the germination phase results in an oxidation reaction to yield the volatile organic compound, which can include an aldehyde.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a schematic of one embodiment of a self-contained biological indicator contemplated by the present invention.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

Generally speaking, the present invention is directed to a growth medium for a biological indicator. The growth medium includes a base growth medium as well as an additive comprising a carbon source unit, such as, but not limited to, glucose, and a volatile organic compound unit. The present invention is also directed to a self-contained biological indicator (SCBI) that includes a container, spores disposed on a carrier, a growth medium, and an additive. The additive can be contained within the growth medium, can be disposed on the spore carrier, or can be disposed on the spores themselves. In one embodiment, the additive includes a carbon source unit, unit such as, but not limited to a carbohydrate or amino acid molecule; or a carbon source molecule containing a part that can form a VOC when reduced or oxidized, such as, but not limited to glucose, and a volatile organic compound unit. Without intending to be limited by any particular theory, the present inventors have found that the addition of the additive to the growth medium facilitates the efficient and accurate detection of a failed sterilization process. In particular, when spores in an SCBI are not killed in a sterilization process, the spores proceed to a germination phase during which an enzyme, such as, but not limited to α-glucosidase, on the spore is activated and reacts with the additive to cleave the volatile organic compound unit from the carbon source unit. Then, the presence of the volatile organic compound, such as at a specified concentration, as measured by methods known by one of ordinary skill in the art, can indicate that the sterilization process in which the SCBI was utilized has failed.

In another embodiment, an additive can be a carbohydrate source such as an amino acid, such as an aliphatic amine. The additive can also be a primary alkyl sulfate ester, or a primary alcohol will be transformed by the action of a cytochrome (e.g.co-enzyme) to form a volatile organic compound. Cytochromes are found on a spore's outer surface and in the initial dormant spore core. They are an intermediary of the respiratory chains and produce the H+ required to transform the NAD and NADP molecules present in abundance in the spores into NADH and NADPH, which are used by the enzymes in the spores to finish the germination and outgrowth process. The outer layers of Bacillus subtilis spore contain one-third of the total spore cytochrome content as well as several enzymes of the electron transport chain (specifically NADH oxidase, dehydrogenase, cytochrome c reductase and NADPH dehydrogenase). In this embodiment, many compounds can be transformed into one group of VOCs, such as aldehydes, thus increasing the sensitivity of detecting a low number of germinating spores by detecting all compounds from a specific chemical group.

In another embodiment, the additive can be modified in a second compound that will be modified again by one or a sequence of reactions into a VOC. Carbon source present in the growth medium will enter the glycolysis and/or Entner-Doudoroff Pathways to form pyruvate. Pyruvate can be fermented in alcohol, ketone, or acid such as 1-butanol, 2,3 butanediol (diacetyl) or propanoate (propanoic acid).

Upon spore germination, the small acid-soluble spore proteins (SASP) serve as a reservoir of amino acids to support protein synthesis. At this stage, SASP utilization is accomplished through the activity of a sequence-specific endoprotease, termed GPR (germination protease), which facilitates SASP degradation.

Some amino acids such as L-alanine, a germination initiator (germinant), is transformed in pyruvate by the alanine dehydrogenase or via the action of the L-glutamate-pyruvate transaminases. Other amino acids will be transformed via other pathways. Valine, leucine, threonine, and isoleucine can be transformed in keto acids such as α-ketoisocrapoic acid (4-methyl-2-oxopentanoic acid). These products can then be transformed in ethanol (VOC), isobutanol (VOC), 2-methyl-1-butanol (VOC), and 3-methyl-1-butanol (VOC).

In any event, the composition of volatile organic compounds allows them to evaporate under average indoor atmospheric temperature and pressure conditions. Several factors influence volatile organic compounds (VOCs) volatility, such as molecular structure, molecular weight, polarity, and intermolecular forces. The volatility decreases as the chain length increases because longer chains have more opportunities for chain-chain interactions via dispersion forces. Also, smaller molecules have fewer intermolecular forces to overcome when transitioning from a liquid to a gas state, so the chain length or the aliphatic substitution end and the pathway/mechanism by which the VOC is produced should be considered. For example, when the chain length or the aliphatic substitution is short as in the aliphatic amino acid sub-group of glycine, alanine, valine, leucine, and isoleucine, there is increased volatility. Regarding threonine, it has a methyl hydroxyl group on one side and a carboxyl group on the other side, so depending on the pathway (reduction/oxidation), both can be converted to a VOC.

Phenylalanine degradation (anaerobic) leads to the formation of L-glutamate and phenylacetate. Phenylacetate can be fermented in Toluene (VOC) and then propanal (VOC). Some amino acid may be reduced by the Strickland reaction, by products are 3-propanoate (3-propanoic acid) derivative.

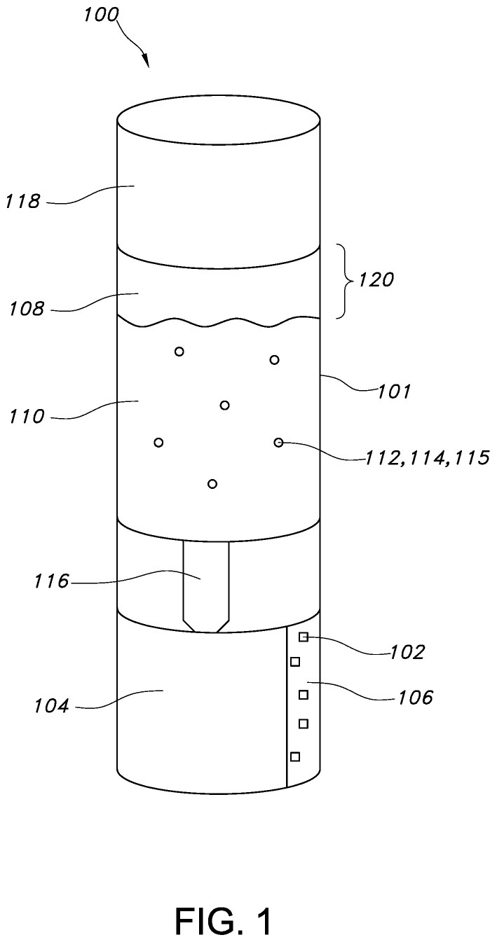

Turning now to FIG. 1, various embodiments of the present invention will be discussed in more detail. FIG. 1 illustrates a self-contained biological indicator 100 that includes a container 101. The container 101 is sealed with a cap 118 and holds a growth medium 110 in an ampoule 108 and a growth chamber 104 that houses a spore carrier 106 containing spores 102. The growth medium 110 can include an additive 112, although it is to be understood that the additive 112 can alternatively or additionally also be present on the spores 102 themselves and/or the spore carrier 106. The additive 112 contains a glucose unit 114 and a volatile organic compound unit 115. The ampoule 108 and the growth chamber 104 are separated by an ampoule crusher 116 that, when activated, is used to introduce the growth medium 110 to the spores 102 after a sterilization cycle has been run. If the sterilization cycle has been successful, then the spores 102 will not germinate. However, if the sterilization cycle has not been successful, the spores 102 will enter the germination phase, during which the spores will release the targeted enzyme or coenzyme. The targeted enzyme or coenzyme then reacts with the additive 112 to cleave the volatile organic compound unit 115 from the carbon unit 114. The volatile organic compound unit 115 is then released into headspace 120 in the ampoule 108. The presence of the volatile organic compound unit 115 in the headspace 120 signals failure of the sterilization cycle and can be collected and analyzed by any suitable methods such as, but not limited to, solid phase microextraction (SPME) and gas chromatography-mass spectrometry (GC-MS).

In some embodiments, the growth medium 110 can include tryptic soy broth, modified soybean casein digest broth, or AGFK (L-asparagine, D-glucose, D-fructose, and K+) germination medium. Further, the SCBI can include spores of Geobacillus stearothermophilus and/or Bacillus atrophaeus.

In addition, in some embodiments, the carbon unit 114 can be an α-glucopyranoside. Additionally, in some embodiments, the volatile organic compound unit 115 can be a functional alkyl, where the alkyl can be methyl, ethyl, propyl, butyl, pentyl, hexyl, heptyl, octyl, nonyl, decyl, undecyl, or dodecyl. Further, the additive 112 that includes the glucose unit 114 and the volatile organic compound unit 115 can be present in the growth medium at a concentration ranging from about 1 millimolar to about 20 millimolar, such as from about 2.5 millimolar to about 15 millimolar, such as from about 5 millimolar to about 10 millimolar.

In some embodiments, the additive 112 can have the following structure:

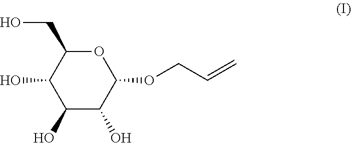

In some embodiments, the additive 112 can have the following structure:

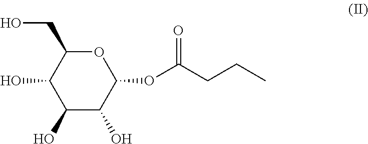 [SMILES: CCCC(=O)O[C@H]1O[C@H](CO)[C@@H](O)[C@H](O)[C@H]1O]

In some embodiments, the additive can have the following structure:

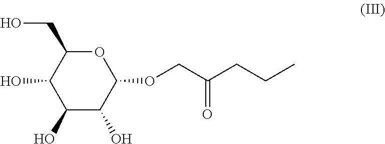 [SMILES: CCCC(=O)CO[C@H]1O[C@H](CO)[C@@H](O)[C@H](O)[C@H]1O]

In some embodiments, the additive can have the following structure:

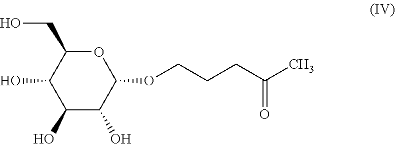 [SMILES: CC(=O)CCCO[C@H]1O[C@H](CO)[C@@H](O)[C@H](O)[C@H]1O]

In some embodiments, the additive can have the following structure:

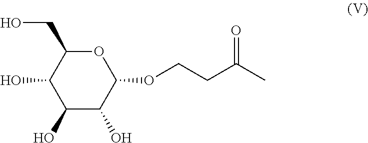 [SMILES: CC(=O)CCO[C@H]1O[C@H](CO)[C@@H](O)[C@H](O)[C@H]1O]

In some embodiments, the additive can have the following structure:

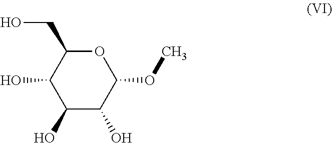

In some embodiments, the additive can have the following structure:

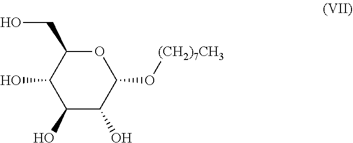

The present invention may be better understood with reference to the following examples.

### EXAMPLE 1

### Objectives

To verify that the addition of specific compounds to a base growth medium will increase the production of VOCs during the first 20 minutes of the germination of Geobacillus stearothermophilus spores.

### Methodology

Spore suspension of Geobacillus stearothermophilus (1×10⁶ CFU/0.1 mL) were diluted to reach a concentration of 500-800 CFU/30-50 μL after being heat shock for 15 min at 95-99° C. to kill all vegetative cell that might have been present in the suspension.

A modified soybean casein digest broth was diluted (10×) with HPLC grade water and sterilize by filtration 0.45 μm to limit the amount of VOC in the background. was used with or without the addition of an additive. The numbers of samples used as well as the combination studied are summarized in Table 1.

TABLE 1
Number of samples to test per combination

| | | With about |
|---|---|---|
| | | 500-800 CFU |
| | Controls | (30-50 μL |
| | (no | of the spore |
| | spores) | suspension) |
| SPME fiber qualification with methanol | 3 | 0 |
| 2500 μg (to be done with each fiber) | | |
| Growth medium alone | 5 | 5 |
| Growth medium + Methyl alpha-D- | 5 | 5 |
| glucopyranoside at 5 mM final | | |
| concentration in growth medium* | | |

For the VOC testing, 0.5 mL of the sterile diluted growth medium was transferred in each VOC collection vials. The vials were prewarm at 60° C. for at least 10 minutes. Before starting the VOC sampling, the additive was added to the vials, then the spores according to Table 1 in function of the test to be performed. The VOC were sampled by inserting the SPME fiber into the vial as soon as the spores were added, and the vials were put back in the temperature control systems. The SPME fiber use was an assembly Divinylbenzene/Carboxen/Polydimethylsiloxane (DVB/CAR/PDMS) (Sigma Aldrich—product number 57328-U from Supelco) conditioned at 270° C. for 30 minutes before use. The SPME fibers were removed 20 minutes after the addition of the spores and were analyzed using the GC-MS according to the lab protocols for the apparatus.

### Results

The α-glucosidase activity should transform the additive into methanol and glucose. Methanol, being a VOC, can be analyzed. Glucose can be used by the spores to produces additional VOC via other enzyme and pathways. In addition to the VOC identification, the intensity of their detection was analyzed. Because there is more VOC produced in the presence of spores, the intensity of one VOC may decrease even if the same quantity is produced. However, an increase in the intensity of a specific VOC in the presence of spore with or without the additive in comparison to the growth medium, indicate that its concentration has increased. The analysis of the VOC produced are complicated by the fact that the spores are not germinating all at the same time. In addition, some compounds may also be used by spores more advance in their germination process if they are soluble in water. Thus, some VOC may be detected only in a few samples. Some of the pathways leading to VOCs may have been identified (Table 2), for some other VOC such as 1-Nonene, Nonane, 1-Octene, Octane, Heptane that were detected only in the presence of the additive, the pathways are not known.

VOCs detected demonstrates that they could be produced by the activity of the cytochrome (aldehyde group VOC- formaldehyde, nonanal, octanal, pentanal, heptanal, hexanal, 2-propenal) by the used of the additive (glucose or methanol liberated by the action of the α-glucosidase activity) for VOC that may have been produced by the fermentation of pyruvate (1-butanol, 2,3-butanedione, propanoic acid) or from methanol (formaldehyde), and/or from other pathways such as the ones from amino acid degradation (formaldehyde, proprionic acid).

TABLE 2
Number of sample testing positive for a specific
VOC with possible identified metabolic pathways.

| VOC | Fiber + | Growth | | |
|---|---|---|---|---|
| (and | Methanol | medium | GM + Spore | GM + S + |
| intensity) | (control) | (GM) | (S) | Additive |
| Methanol | 5+/5 | 5+/5 | 5+/5 | 5+/5 |
| | | | (−2X to 5X)* | (−2X to 5X) |
| Propene | 0+/5 | 0+/5 | 5+/5 | 5+/5 |
| Formaldehyde | 0+/5 | 4+/5 | 4+/5 | 2+/5 |
| | | | (+2X) | (+10X) |
| 1-butanol | 3+/5 | 4+/5 | 5+/5 | 4+/5 |
| | | | (+up to 3X) | (+up to 3X) |
| 2,3-butandiol | 0+/5 | 0+/5 | 0+/5 | 2+/5 |
| Propanoic | 0+/5 | 0+/5 | 1+/5 | 2+/5 |
| acid | | | | |
| Nonanal | 5+/5 | 5+/5 | 5+/5 | 5+/5 |
| Octanal | | | (up to 3X) | (up to 3X) |
| Heptanal | | | | |
| Acetaldehyde | 3+/5 | 4+/5 | 4+/5 | 5+/5 +up |
| | | | | to 2X |

*In comparison to the growth medium intensity

Some examples of the possible pathways leading to VOC are represented below. Propene is clearly coming from the spores germinating and its concentration is not increasing from the presence of the additive. Propene may be produced by the reaction of a fatty acid with hydrogen peroxide or an acyl-[acy-carrier-protein] with malonyl CoA+a reduced electron carrier+H+ as described in (VIII).

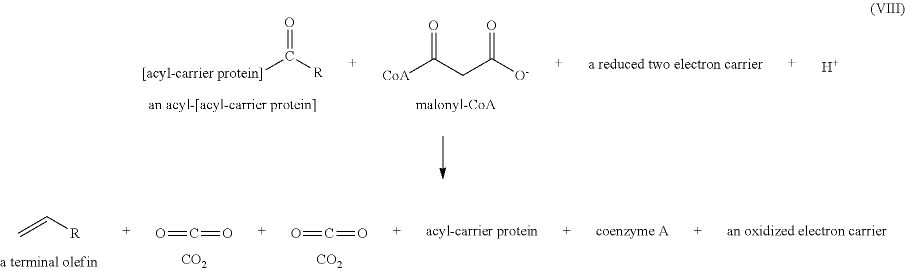

Formaldehyde can be produced from methanol (IX) or by the action of the cytochrome as other aldehyde (X).

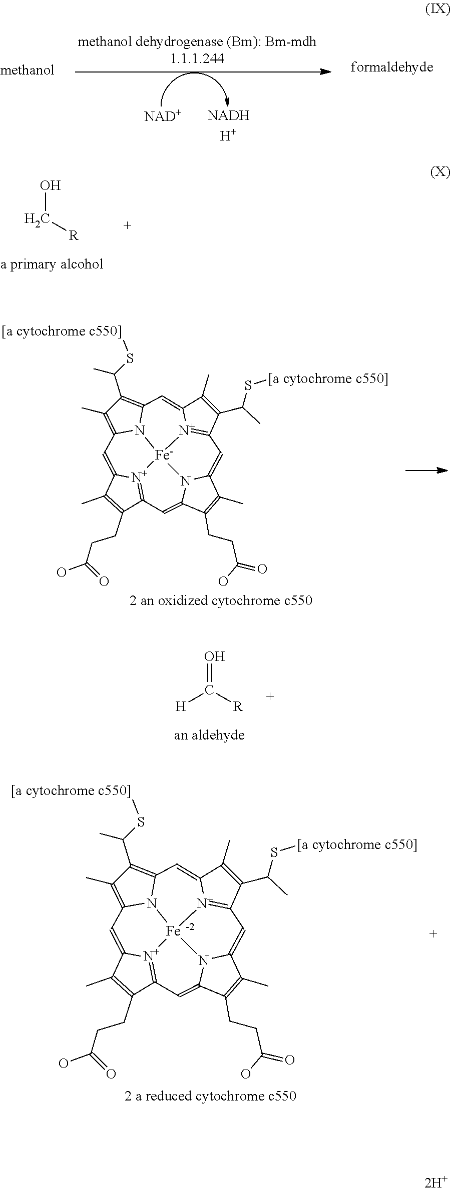

2,3 butandione may be produced by the fermentation of pyruvate to acetoin (XI), and since the transformation to acetoin need NADH, it might accumulate in the spore/medium. The spontaneous reaction will happen in aerobic condition.

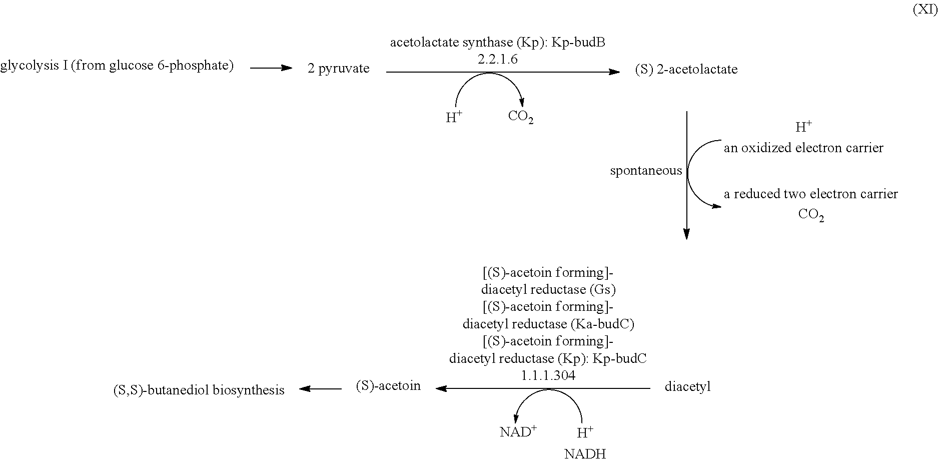

It can also happen from a generic reaction (XII)

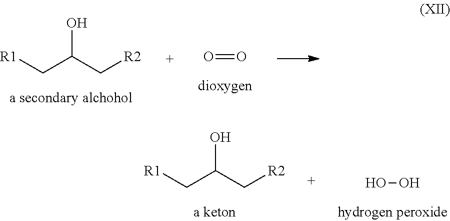

Propanoic acid may be produced by pyruvate fermentation or degradation of L-Threonine (XIII) or other amino acid such as L-valine.

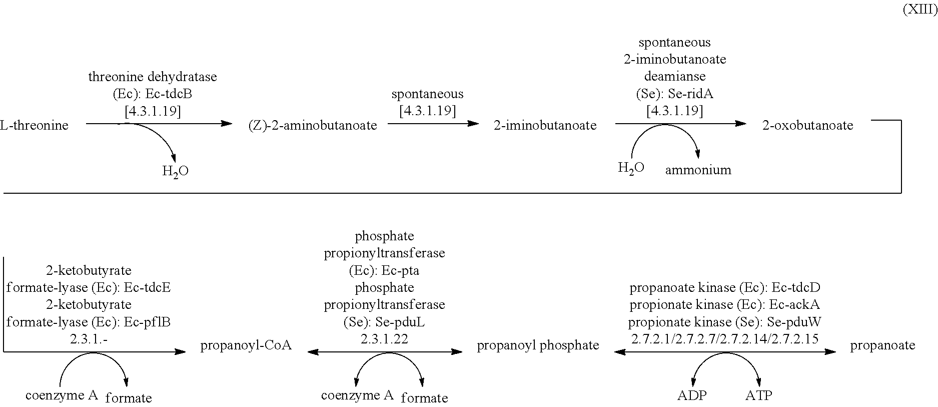 [SMILES: O ; O ; "coenzyme A" ; "coenzyme A" ; "ADP" ; "ATP"]

Aldehydes such as nonanal, octanal, hexanal, or heptanal may be produced by the transformation of aliphatic amine by cytochrome (XIV), primary alkyl sulphate ester (XV).

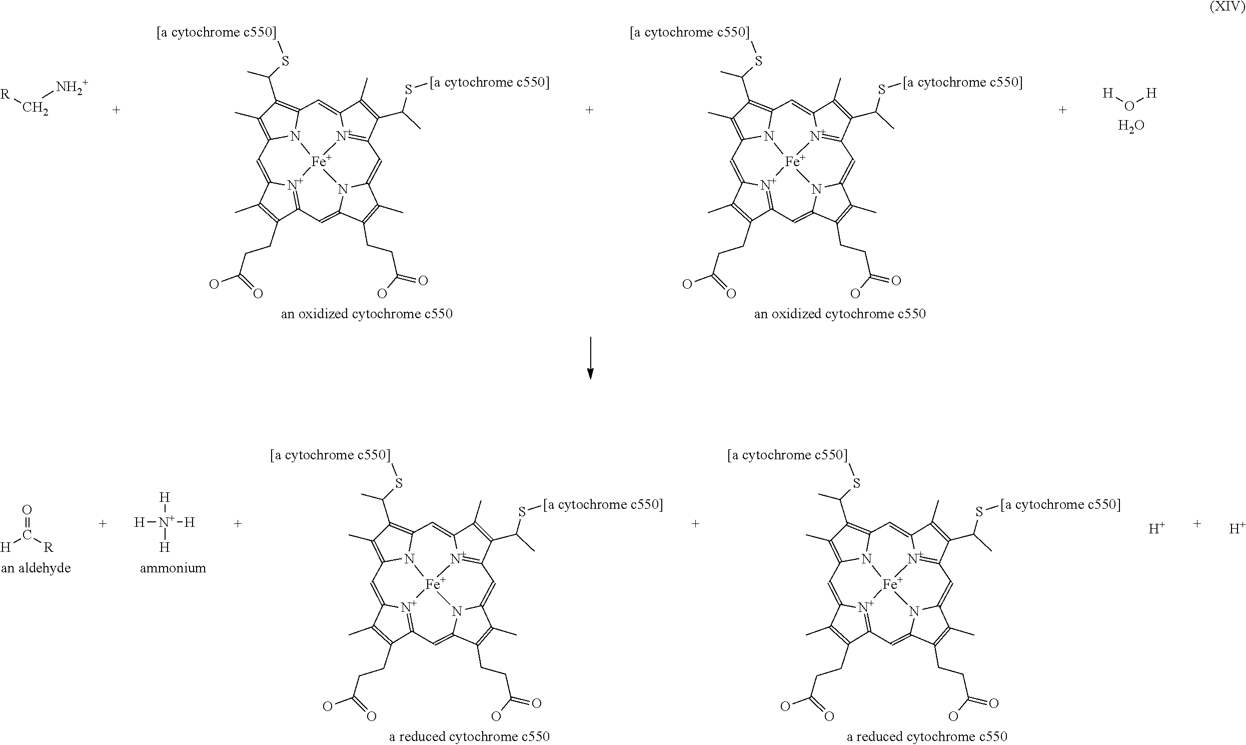

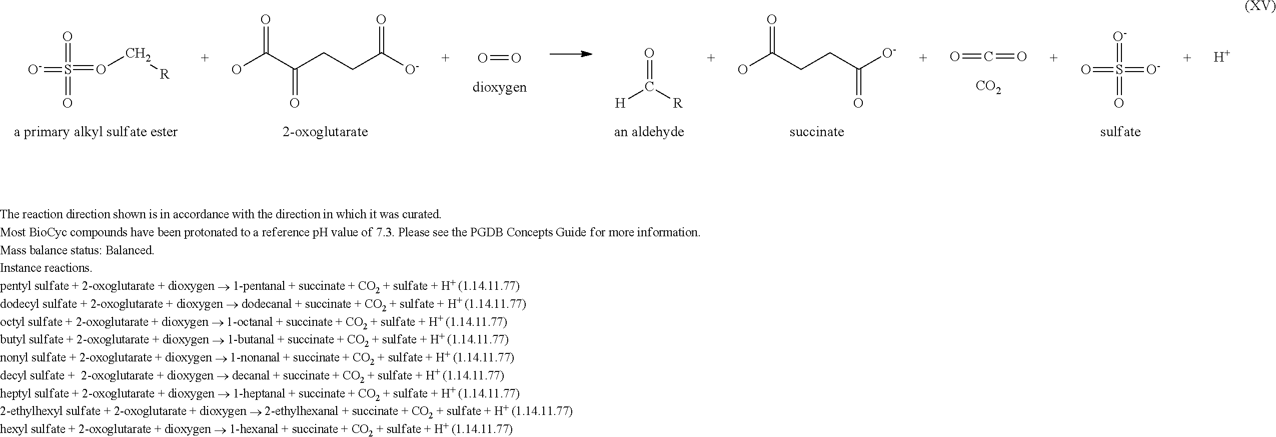

### EXAMPLE 2

### Objectives

To verify that the addition of specific compounds to a germination medium (permitting germination but limiting the outgrowth) increase the production of VOCs during the first 15 minutes of the germination of Geobacillus stearothermophilus spores.

### Methodology

Spore suspension of Geobacillus stearothermophilus (1×10⁶ CFU/0.1 mL) were heated for 30 min at 99-100° C. to kill all vegetative cell that might have been present in the suspension.

A growth medium composed of a modified soybean casein digest broth diluted (200×) with the sterilized germination medium (1,0 mM L-valine in 10 mM sodium phosphate buffer (pH 8.0) as described by “thou T, Dong Z, Setlow P, Li Y-q (2013), Kinetics of Germination of Individual Spores of Geobacillus stearothermophilus as Measured by Raman Spectroscopy and Differential Interference Contrast Microscopy. PLoS ONE 8(9): e74987)” was used with or without the addition of L-threonine or glucose at 5 mM. One test was also performed with the germination medium by itself, without the modified soybean casein digest broth diluted (200×) and the additives. The controls included the SPME fiber alone, in the presence of 2500 μg methanol, and growth medium with and without the L-Threonine or glucose.

For the VOC testing, 0.5 mL of the control solution (methanol) or sterile growth medium was transferred in each VOC collection vials. The vials were prewarm at 65° C. for at least 10 minutes. Before starting the VOC sampling, the L-threonine or glucose 5 mM was added to the vials, then the 5000-10000 spores (except for the controls). The VOC were sampled by inserting the SPME fiber into the vial as soon as the spores were added, and the vials were put back in the temperature control systems. The SPME fiber use was an assembly Divinylbenzene/Carboxen/Polydimethylsiloxane (DVB/CAR/PDMS) (Sigma Aldrich—product number 57328-U from Supelco) conditioned at 270° C. for 30 minutes before use. The SPME fibers were removed 15 minutes after the addition of the spores and were analyzed using the GC-MS according to the lab protocols for the apparatus.

### Results

The L-threonine should lead to the formation of carbon dioxide, proprionic acid, acetic acid, and other volatile fatty acids, whereas glucose will lead to acetate (anaerobic respiration) or pyruvate, lactic acid or butandiol (fermentation).

In this example, VOCs coming from the growth medium itself were less predominant than in Example 1. It also demonstrates that not only the VOC link to the glucose molecule can be detected, but also VOC produced using glucose itself.

The addition of L-threonine or glucose leads to the formation of some common VOC such as 1-nonene but also different VOC as shown in Table 3.

TABLE 3
VOC produced in the presence of the L-threonine and or
glucose in the presence of spore (qualitative amount).

| | | Growth | | |
|---|---|---|---|---|
| | Gemination | medium | GM + | GM + |
| VOC Name | medium | (GM) | L-threonine | glucose |
| Acetaldehyde | <control | <control | ++ | <control |
| 1-Butanol | Not | Not | ++ | Not |
| | detected | detected | | detected |
| Butanal | ++ | <control | <control | +++ |
| 1-nonene | Not | Not | ++ | ++ |
| | detected | detected | | |
| Benzene, 1-ethenyl- | <control | <control | + | + |
| 4-ethyl- | | | | |
| Benzaldehyde, | +++ | Not | ++ | Not |
| 3-ethyl- | | detected | | detected |
| Cyclopentanone | <control | Not | Not | + |
| | | detected | detected | |
| 1-Octene | Not | Not | Not | ++ |
| | detected | detected | detected | |
| Propanal, 2-methyl | ++ | Not | ++ | Not |
| | | detected | | detected |
| Propene | Not | Not | + | +++ |
| | detected | detected | | |
| Pyrazine, 2,5- | Not | Not | +++ | Not |
| dimethyl- | detected | detected | | detected |
| 2,4,6-Trimethyl-1- | Not | Not | ++ | Not |
| nonene | detected | detected | | detected |
| Benzoic acid, 4-(4- | Not | Not | ++ | Not |
| propylcyclohexyl)-, | detected | detected | | detected |
| 4′-cyano[1,1′- | | | | |
| biphenyl]-4-yl ester | | | | |

Some examples of the possible pathways leading to VOC are represented below. Propene was detected in example 1, this example is demonstrating that propene is link to the used of carbohydrate compounds such as glucose present in the growth medium or added into it.

Glucose may be fermented to 1-butanal by Clostridium sp. (Biocycle Pathway PWY-6594, www.biocyc.org). Bacillus sp. have some of the same enzymes as Clostridium sp. when grown under anaerobic conditions (fermentation).

Threonine may be degraded in acetaldehyde by Bacillus subtilis or Clostridium pasteurianum via a L-threonine aldolase (XVI). See www.Biocyc.org. Threonine degradation Pathway 4). Threonine may also be fermented in 1-butanol via pyruvate (XVI).

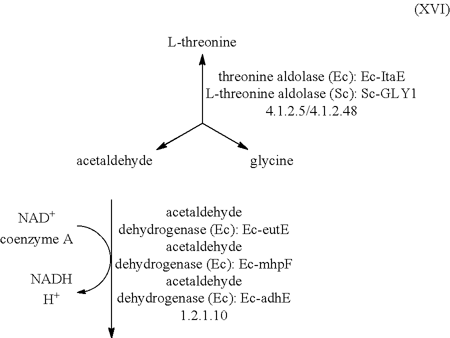

Other VOCs such as Benzaldehyde, 3-ethyl- are known to be produced by Clostridium sp. (C. A. Rees, A. Shen, and J. E. Hill. 2016. Characterization of the Clostridium difficile volatile metabolome using comprehensive two-dimensional gas chromatography time-of-flight mass spectrometry. Journal of Chromatography B Vol. 1039 Pages 8-16.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A growth medium for a biological indicator, the growth medium comprising:
a base growth medium; and
an additive comprising a carbon source unit and a volatile organic compound unit.

2. The growth medium of claim 1, wherein the base growth medium comprises tryptic soy broth, modified soybean casein digest broth, or AGFK (L-asparagine, D-glucose, D-fructose, and K+) germination medium.

3. The growth medium of claim 1, wherein the carbon source unit comprises an α-glucopyranoside and the volatile organic compound unit comprises a functional alkyl.

4. The growth medium of claim 1, wherein additive has the following structure:

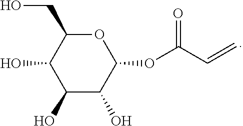 [SMILES: C=CC(=O)O[C@H]1O[C@H](CO)[C@@H](O)[C@H](O)[C@H]1O]

5. The growth medium of claim 1, wherein the additive has the following structure:

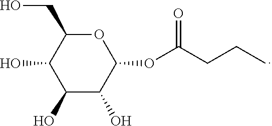 [SMILES: CCCC(=O)O[C@H]1O[C@H](CO)[C@@H](O)[C@H](O)[C@H]1O]

6. The growth medium of claim 1, wherein the additive has the following structure:

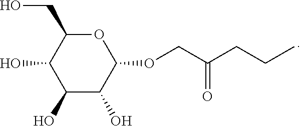 [SMILES: CCCC(=O)CO[C@H]1O[C@H](CO)[C@@H](O)[C@H](O)[C@H]1O]

7. The growth medium of claim 1, wherein the additive has the following structure:

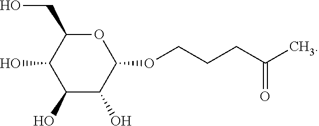 [SMILES: CC(=O)CCCO[C@H]1O[C@H](CO)[C@@H](O)[C@H](O)[C@H]1O]

8. The growth medium of claim 1, wherein the additive has the following structure:

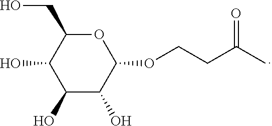 [SMILES: CC(=O)CCO[C@H]1O[C@H](CO)[C@@H](O)[C@H](O)[C@H]1O]

9. The growth medium of claim 1, wherein the additive has the following structure:

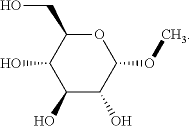

10. The growth medium of claim 1, wherein the additive has the following structure:

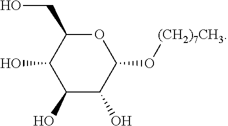

11. The growth medium of claim 1, wherein the additive is present in the growth medium at a concentration ranging from about 1 millimolar to about 20 millimolar.

12. A self-contained biological indicator comprising:
a container;
spores disposed on a carrier;
a growth medium; and
an additive, wherein the additive comprises (1) a carbon source unit or (2) a carbon source molecule containing a part that forms a volatile organic compound when oxidized, and (3) a volatile organic compound unit.

13. The self-contained biological indicator of claim 12, wherein the self-contained biological indicator comprises spores of Geobacillus stearothermophilus or Bacillus atrophaeus.

14. The self-contained biological indicator of claim 12, wherein the growth medium comprises tryptic soy broth or modified soybean casein digest broth.

15. The self-contained biological indicator of claim 12, wherein the carbon source unit comprises an α-glucopyranoside, the carbon source molecule comprises glucose, and the volatile organic compound unit comprises a functional alkyl.

16. The self-contained biological indicator of claim 12, wherein the additive has the following structure:

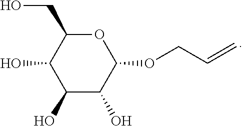

17. The self-contained biological indicator of claim 12, wherein the additive has the following structure:

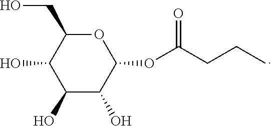 [SMILES: CCCC(=O)O[C@H]1O[C@H](CO)[C@@H](O)[C@H](O)[C@H]1O]

18. The self-contained biological indicator of claim 12, wherein the additive has the following structure:

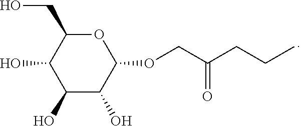 [SMILES: CCCC(=O)CO[C@H]1O[C@H](CO)[C@@H](O)[C@H](O)[C@H]1O]

19. The self-contained biological indicator of claim 12, wherein the additive has the following structure:

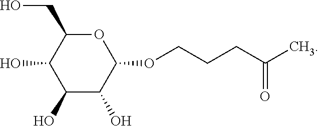 [SMILES: CC(=O)CCCO[C@H]1O[C@H](CO)[C@@H](O)[C@H](O)[C@H]1O]

20. The self-contained biological indicator of claim 12, wherein the additive has the following structure:

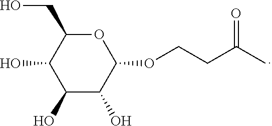 [SMILES: CC(=O)CCO[C@H]1O[C@H](CO)[C@@H](O)[C@H](O)[C@H]1O]

21. The self-contained biological indicator of claim 12, wherein the additive has the following structure:

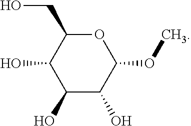

22. The self-contained biological indicator of claim 12, wherein the additive has the following structure:

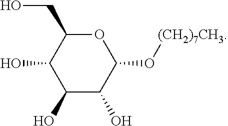

23. The self-contained biological indicator of claim 12, wherein the additive is present in the growth medium at a concentration ranging from about 1 millimolar to about 20 millimolar.

24. The self-contained biological indicator of claim 12, further comprising a growth medium ampoule and an ampoule crusher.

25. The self-contained biological indicator of claim 12, wherein release of α-glucosidase as a result of the spores being in a germination phase results in the cleavage of the volatile organic compound unit.

26. A growth medium for a biological indicator, the growth medium comprising:
a base growth medium; and
an additive comprising a carbohydrate source that leads to formation of a volatile organic compound by an action of an enzyme or a coenzyme that is active in a germination phase of spores that are introduced to the growth medium.

27. The growth medium of claim 26, wherein the release of cytochrome c reductase as a result of spores that have been introduced to the growth medium being in the germination phase results in an oxidation reaction to yield the volatile organic compound.

28. The growth medium of claim 27, wherein the volatile organic compound comprises an aldehyde.

29. A self-contained biological indicator comprising:
a container;
spores disposed on a carrier; and
a growth medium, wherein the self-contained biological indicator includes a carbohydrate source that leads to formation of a volatile organic compound by an action of an enzyme or a coenzyme that is active in a germination phase of spores that are introduced to the growth medium.

30. The self-contained biological indicator of claim 29, wherein the carbohydrate source comprises an amino acid.

31. The self-contained biological indicator of claim 29, wherein the carbohydrate source comprises an aliphatic amine.

32. The self-contained biological indicator of claim 29, wherein the carbohydrate source is disposed within the growth medium, on the carrier, or on the spores.

33. The self-contained biological indicator of claim 29, wherein the release of cytochrome c reductase as a result of the spores that have been introduced to the growth medium being in the germination phase results in an oxidation reaction to yield the volatile organic compound.

34. The self-contained biological indicator of claim 33, wherein the volatile organic compound comprises an aldehyde.
